(19) United States
(12) Patent Application Publication
(10) Pub. No.: US 2005/0027565 A1
(43) Pub. Date: Feb. 3, 2005

(54) Motivational method for helping substance abusers quit substance abuse

(76) Inventor: John Sader, St-Lambert (CA)

Correspondence Address: SEYFARTH SHAW, 55 EAST MONROE STREET, SUITE 4200, CHICAGO, IL, 60603-5803, US

(21) Appl. No.: 10/876,138

(22) Filed: Jun. 23, 2004

(30) Foreign Application Priority Data
Jun. 23, 2003 (CA) 2,433,136

(51) Int. Cl. G06F 17/60; A61K 31/497

(52) U.S. Cl. 705/2; 705/1

(57) ABSTRACT

The present invention discloses a personalized method of health-related behavior change that stimulates the limbic emotional system in order to reinforce the positive emotions and drives that already reside there as well as the cognitive component of what the ideal self wishes to be. In this sense the method strives to direct it's message to both positive emotions as well as the basic human values and enhance and consolidate preexisting motivations. The method may also provide for the promotion of health related products and services.

### TECHNICAL FIELD

This invention generally relates to a motivational method for helping substance abusers quit substance abuse, more particularly tobacco, alcohol, food or even drugs. This invention further relates to a motivational method for helping a person modify their behavior which they may want to modify, such as for example excessive eating, or their unwanted or unhealthy habits.

### BACKGROUND

One of the most common sort of substance abuse encountered is smoking, although other sorts of substance abuse, such as alcohol, drugs food, are also common.

Previous attempts to modify behavior, such as the display of an informational visual anti-smoking image to a user fail to provide an enduring motivational message to a smoker to avoid further substance abuse. As well, previous attempts to substitute various devices for a smoker's cigarettes also have failed in their efforts to eliminate substance abuse as they require a smoker to immediately cease smoking without any psychological encouragement to do so and are particularly difficult to convince a smoker to utilize. Similarly, attempts to replace cigarette packaging with other forms of containers and attempts to electrically shock a smoker upon each use of a cigarette also fail to eliminate substance abuse in view of the likelihood that a smoker will simply avoid such packaging and such devices altogether. In particular, because all of these aforementioned attempts can be easily avoided by the substance abuser, such prior attempts have been particularly ineffective.

More recent attempts rely on eliciting fear in the smoker, this as for effect the stimulation of the limbic system in the brain, which is the emotional center, and is counterproductive as the same fear that hopes to scare the smoker into stopping may overflow to the smoker's other fears, such as fear of quitting, of being obliged to manage his/her emotions, of not succeeding to quit, etc. This may thus have the effect of increasing these fears and indirectly increasing the emotional drive to smoke.

It is important to understand that previous attempts were aimed at enticing smokers to stop smoking rather than at smokers that were already motivated to do so.

Accordingly, it is an object of the present application to obviate or mitigate some or all of the above disadvantages.

### SUMMARY

According to an embodiment of the invention, there is provided a motivational method for helping substance abusers who have already decided to quit to overcome their addiction to an addictive substance, comprising the steps of selecting a personalized motivational message from a pre-selected group of messages posted on a web site, printing said chosen motivational message and applying said printed motivational message onto an addictive substance's container, wherein the motivational message enhances an already existing motivation for said substance abuser to quit its substance abuse.

In another aspect of the invention, there is provided a motivational method for helping substance abusers who have already decided to quite substance abuse to overcome their addictions, comprising the steps of choosing a label comprising a personalized motivational message and applying the label onto an addictive substance's container, wherein the motivational message enhances and consolidates an already existing motivation to quit substance abuse.

In a further aspect of the invention, there is provided a motivational method for helping substance abusers who have already decided to quite substance abuse to overcome their addictions, comprising the steps of choosing cut-out container comprising a personalized motivational message and replacing the addictive substance's container with the cut-out container, wherein the motivational message enhances and consolidates an already existing motivation to quit substance abuse.

In a further still aspect of the invention, there is provided a motivational method for helping substance abusers who have already decided to quite substance abuse to overcome their addictions, comprising the steps of choosing a personalized motivational message, printing the chosen motivational message on a cut-out container and replacing the addictive substance's container with the cut-out container, wherein the motivational message enhances and consolidates an already existing motivation to quit substance abuse.

In a yet further aspect of the invention, there is provided a motivational method for helping substance abusers who have already decided to quite food abuse to overcome their addictions, comprising the steps of choosing a motivational message, printing the chosen motivational message on a magnet and applying the magnet to a refrigerator door, wherein the motivational message enhances and consolidates an already existing motivation to quit food abuse.

In a yet further still aspect of the invention, there is provided a web-based method for individual tailoring aid to a person wishing to cease the use of an addictive substance packaged in a container, the method comprising the steps of accessing by the person a web based server located at a remote location, the server comprising at least one group of motivational messages pre-selected by the web server administrator, selecting by the person from the group of motivational messages at least one message suited to the particular needs and goals of the person, printing by said person said at least one motivational message at a location selected by the person and affixing by the person the printed motivational message onto the container such that the printed motivational message is visible to the person and wherein the motivational message enhances an already existing motivation for the person to cease using the addictive substance.

In a another still aspect of the invention, there is provided a system to assist a person wishing to cease the use of an addictive substance packaged in a container, said system comprising a server-based, internet accessible data base comprising at least one motivational message formatted to be selected and printed by the person so that the motivational message can be affixed to the container, a communication device which allows the person to remotely access the data base and a printing device on which the motivational message can be printed.

### BRIEF DESCRIPTION OF THE FIGURES

Embodiments of the invention will now be described by way of examples only with reference to the accompanying drawings, in which:

FIG. 1 is a schematic view of the main components of an on-line system in accordance with a particular embodiment of the present invention.

FIG. 2 is a view of a set of labels in accordance with a particular embodiment of the present invention.

FIG. 3a to 3c are schematic views of the steps to follow to apply the set of labels of FIG. 2 unto a cigarette pack.

FIG. 4 is a view of a set of labels in accordance with a further embodiment of the present invention.

FIG. 5a is a view of the front of a set of cutouts in accordance with still a further embodiment of the present invention.

FIG. 5b is a view of the back of the set of cutouts shown in FIG. 5a

FIG. 6 is a detail of a label as shown in FIG. 1 or a cutout as shown in FIGS. 5a and 5b.

FIG. 7 is an alternate detail of a label as shown in FIG. 1 or a cutout as shown in FIGS. 5a and 5b.

### DETAILED DESCRIPTION

Embodiments of the method according to the present invention are to be used by, preferably, individuals who have already decided to stop substance abuse, for example smoking, and who have ideally chosen a quit-date. It should be noted that the method may be applied to other addictions, such as, for example, alcohol, drug, food addiction.

Taking the example of a smoker wishing to quit smoking, typically once the smoker has chosen a quit-date he/she is told to prepare himself/herself in the interim to quit smoking. Unfortunately most smokers do not go through any sort of pre-quit program in order to better prepare themselves for the challenge. The method is intended to consolidate the motivation of the smoker already prepared to pass into the action phase, that is quitting smoking, but not yet at his/her quit-date. Consolidating the person's own chosen motivators reinforces his/her ability to resist dissociating his/her cognitive desires and emotional wants and needs during the brief periodic moments of a cigarette craving. During this instant the smoker is reminded, or more precisely, reminds himself/herself (by having chosen his/her own motivational goals at the outset) why he/she wants to stop being a smoker. In fact, the chosen motivational messages are chosen in such a way that they place the smoker in a position for the accomplishment of a challenge or goal; in this way, refusing that particular cigarette provides the reinforcing pleasure of overcoming a challenge and of the investment of self. This pause, along with a conscious effort to stop and appreciate those relationships that he/she has chosen to prioritize, decreases the power that the craving exerts over him/her. In fact, the craving can be perceived as a need to truly connect with those privileged relationships. The cigarette provides generally only an interim and incomplete form of connection that by definition can only incompletely satisfy the smoker hence the need to continually repeat the process of smoking. The cigarette provides a way of feeling as if one is connected without having to do any of the work or take any of the risks needed to be truly connected to one's self and through the self to others.

The method is a personalized one in which the end-user is not subjected to someone else's messages about quitting smoking but instead chooses his/her own motivational messages from different grouping of messages such as, for example, health, social, youth, family, pregnancy and spirituality, that most reflect his/her desires and drives.

In a particular embodiment, the method uses labels to be apposed to containers, such as cigarette packs, or cut-out replacement packs. The labels or cutouts are inscribed with the personalized messages which may be chosen by the user and are meant to be used typically for a three to four week total period in between the time that the smoker has chosen to quit smoking and the actual time that he/she hits his/her quit-date. As well, the smoker will typically change the labels or cut-outs for ones with different messages every so often, for example every three to four days over the said total period. In an alternate embodiment, the labels or cut-outs may be blank, the motivational messages may then be printed on smaller labels that may be apposed directly on the covering labels or cutouts.

It is important to understand that smokers using the present method have already decided to make a quit attempt and they are already motivated to stop. As opposed to other methods where smokers were to be enticed to become motivated, the smokers targeted by this method are already motivated and the method aims to enhance and consolidate that already preexisting motivation. It is thus preferable that the labels be applied by the smokers themselves.

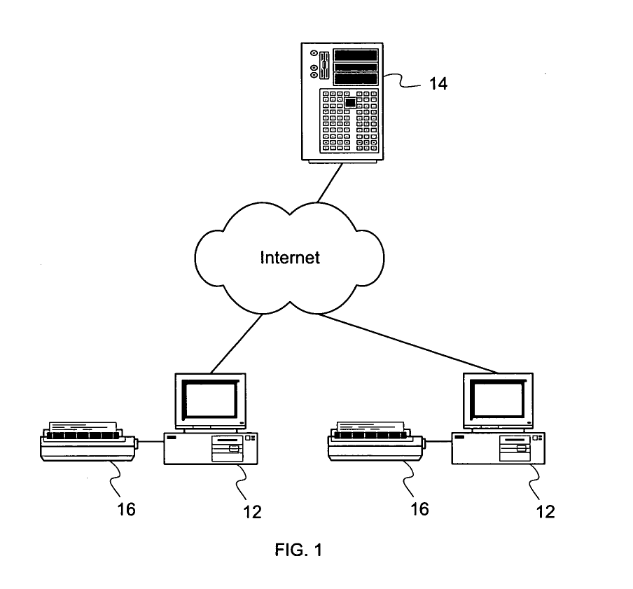

In accordance with a particular embodiment of the present invention, the motivational messages may be created or selected with the use of a program on a personal computer (12), either remotely from the Internet, as shown in FIG. 1, such as from a web-site server or database (14), or locally from a diskette, CDROM or other such distribution means, and then printed unto a desired medium, such as labels, on a printer (16). The personal computer (12) may also be any communication device such as, for example, a laptop computer, a cellular phone or a personal digital assistant. In the situation that a person does not have the necessary computer hardware and printer to get access to the downloadable version, he/she may order or acquire them at a place of business with an interest in providing such a service to its clientele. These may be pre-printed versions personalized according to the smoker's choice, with the selected motivational messages already printed on labels or cut-outs.

Besides being a positive motivational tool, the method may also double as a promotional tool. This is accomplished by the sponsorship of the labels or cut-outs by, for example, ‘healthy living’ type enterprises. The sponsorship may take the form of an enterprise logo or message which is added to the labels or cutouts.

These ‘healthy living’ enterprises may be readily identified by the smoker as being pro-health, pro-being, pro-doing and as such represent oft chosen replacement pleasures by the smoker wanting to stop being a smoker. Often the smoker chooses to get-in-shape, to eat better, to travel, to communicate more etc. Examples of such enterprises are pharmaceutical companies, vitamin companies, sports equipment companies, pharmacies, insurance companies, communication companies, healthy food companies, travel companies and others who are perceived as clean, autonomy-oriented enterprises. Other companies or organizations of interest as sponsors could include government health departments, spirituality-based groups and social-cause groups both for-profit and not-for-profit.

These companies already do a great deal of marketing that vehicle their enterprise as a healthy person enterprise and their sponsorship of the labels implicitly include another positive motivator directly upon the present method.

The promotional aspect of these labels goes even further as they may be provided free-of-charge, for example over the Internet or in a magazine, such that the labels or cutouts become a publicity tool for the enterprises involved with the method. This is so in as such as they associate themselves with a positive motivational product provided freely by the enterprise and conceived to help strengthen the individual who makes use of it. Furthermore, the visitors to the website may augment the traffic and visibility of the website, as well as providing an opportunity to collect consumer data.

The interest of the cigarette package as a promotional tool stems from the fact that the smoker is intended to typically carry the tool with him/her for a period of three to bur weeks and expose himself/herself to the sponsoring companies logo or message whenever he/she takes a cigarette out of the package as well as during the smoking of said cigarette.

For example, for a pack-a-day smoker this means exposing the smoker to the sponsoring company's logo or message typically for about two to four hours per day for three to four weeks. The company thus reinforces its perception as having a supporting role in helping to motivate the smoker to succeed in stopping smoking. As well, smokers usually having more and more to smoke in specifically designated areas, the tool is exposed to other smokers and thus promotes itself.

It should be noted though that seeing an overly-present overly-large logo or message may defeat the purpose of the method as it could be wrongly perceived as primarily a marketing tool rather than the preferred perception of the method as primarily a consolidating motivational method. Accordingly, in a further embodiment, the logos or promotional messages may be understated and kept to a relatively small size on the label as not to diminish the impact of the method itself.

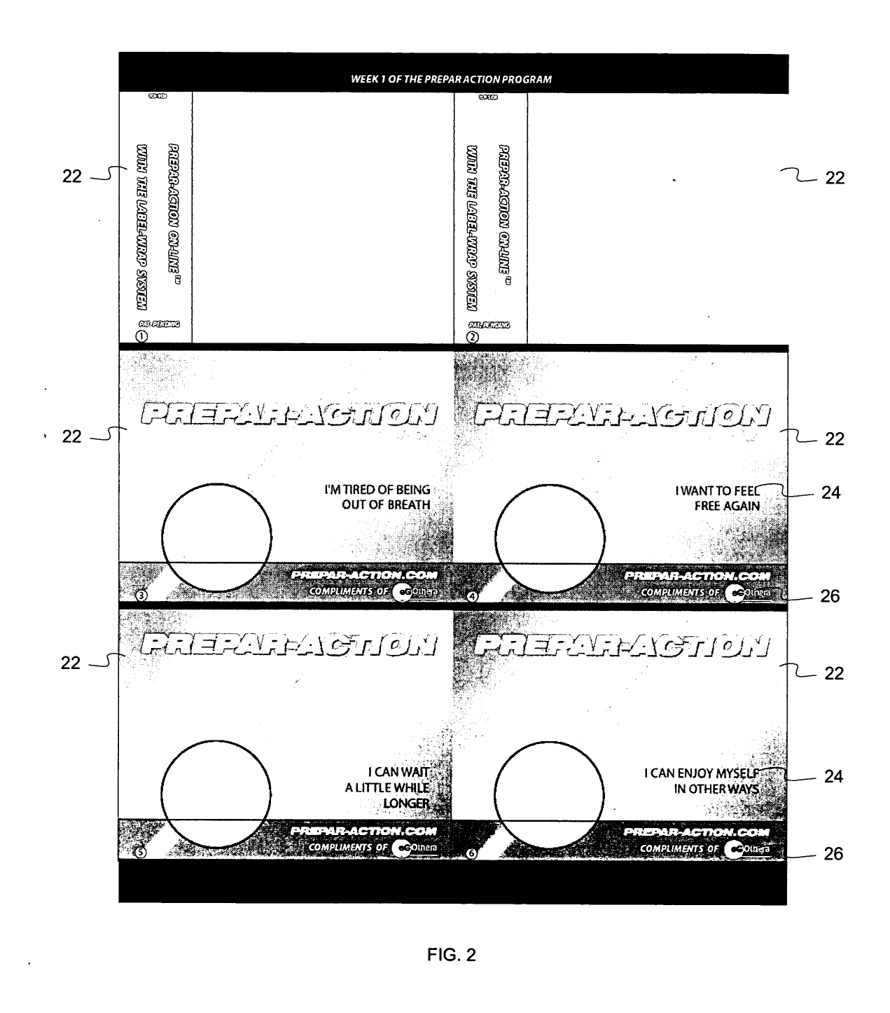

Referring to FIG. 2, there is shown a set of labels (22), not to scale, in accordance with a particular embodiment of the present invention, in which a replacement cover for a Canadian cigarette pack, either regular or king size, is created by apposing a number of labels (22) onto a cigarette pack in order to completely obliterate the front and back covers, sides, top and bottom of the cigarette pack. The sample labels (22) contain motivational personalized messages (24) and sponsoring company logo and message (26).

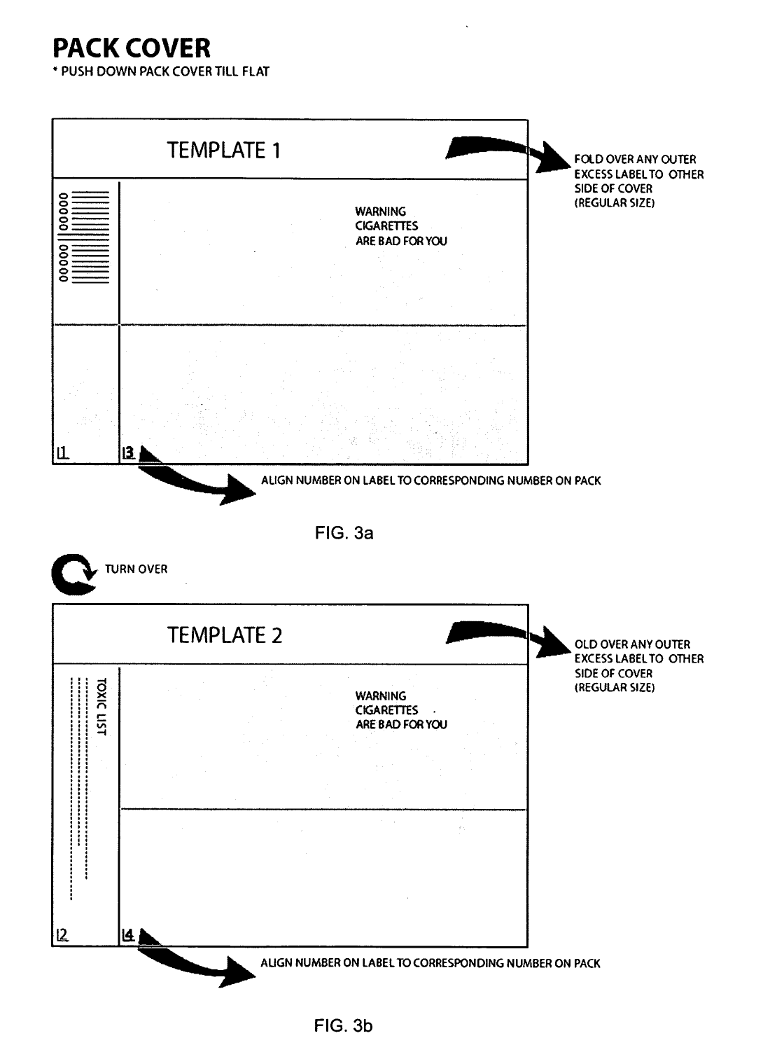

FIG. 3a to 3c illustrate an example of steps 1 to 9 to follow in order to apply the set of labels of FIG. 2 unto a Canadian cigarette pack.

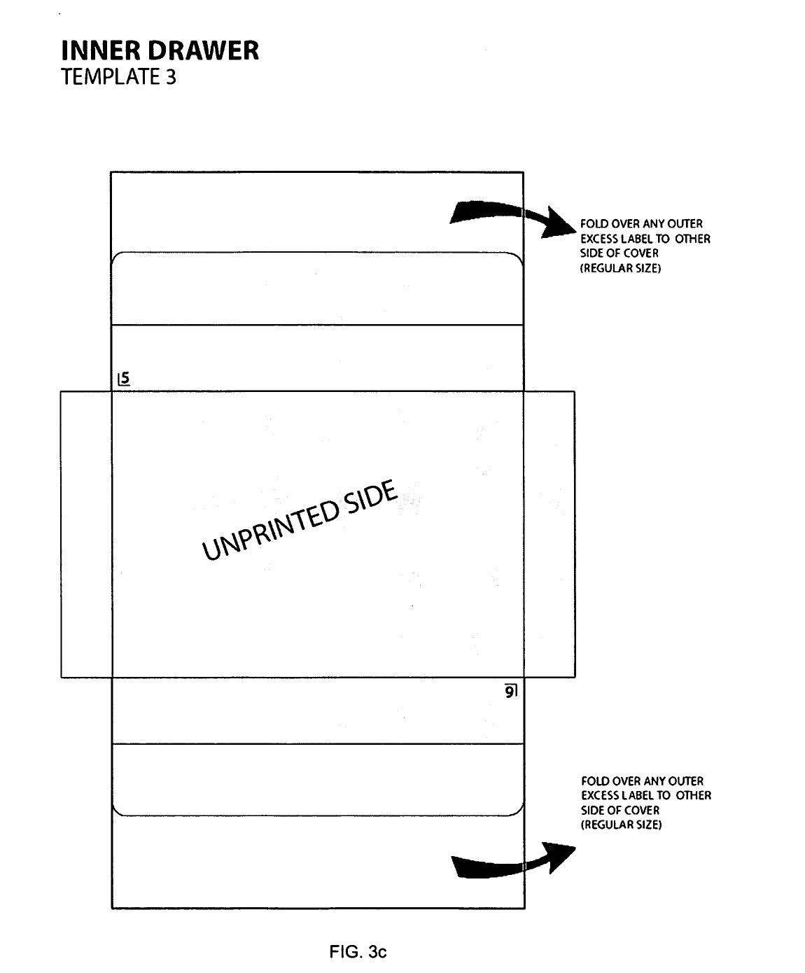

Referring now to FIG. 4, there is shown a set of labels (42), not to scale, in accordance with a further embodiment of the present invention, in which a replacement cover for a US cigarette pack is created by apposing a number of labels (42) onto a cigarette pack in order to completely obliterate the front and back covers and sides of the cigarette pack. The sample labels (42) contain motivational personalized messages (44) and sponsoring company logo and message (46).

The labels (22, 42) may be designed so as to take advantage of one of the most common computer label formats available: the four inch by three inch shipping-size label readily available from a number of companies. When apposed to the cigarette pack, labels (22, 42) of that format completely obscure the front and back covers, the sides and the top and bottom of the Canadian cigarette packs and the front and back covers and sides of in the US cigarette pack, respectively. For a typical United States cigarette pack, two labels (42) of that format are required to completely obliterate the front and back covers and sides of the pack and replace it with the new pack design. As for a typical Canadian pack, six labels (22) of that format are necessary in order to achieve complete coverage.

Of course, other sizes of labels may be used so that a lesser number of labels may be required in order to achieve complete coverage of either the Canadian or US packs or to obliterate only part or certain specific areas of the packs. Special die-cut labels may also be designed in order to completely cover typical Canadian packs with only one label, and the same may be accomplished with typical American packs as well.

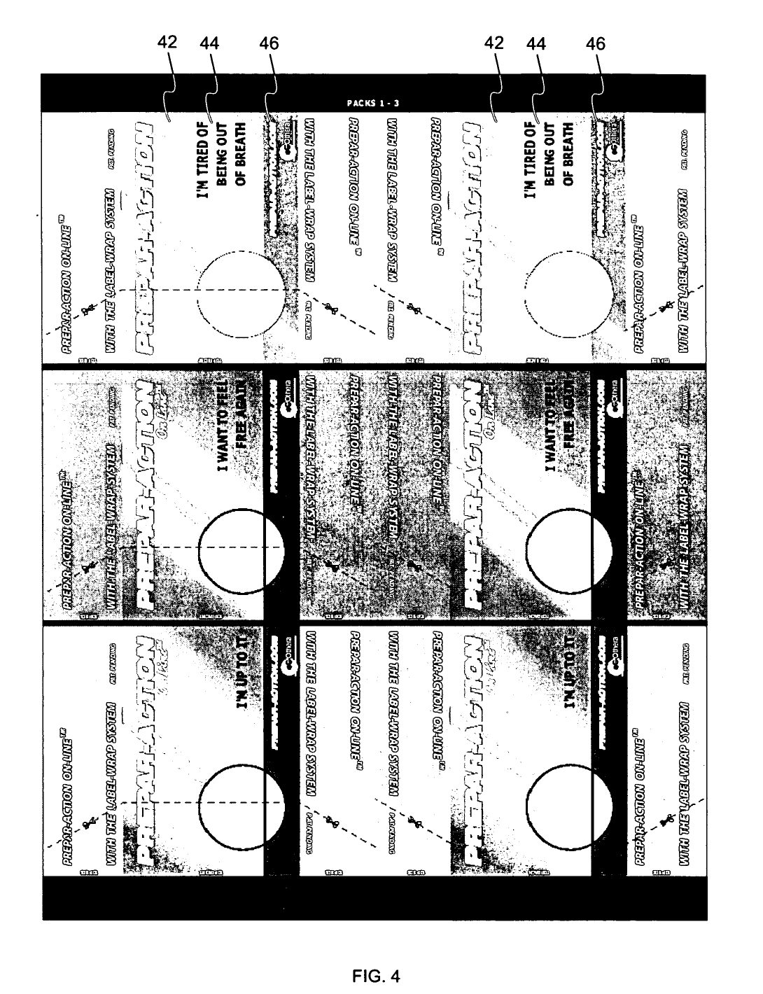

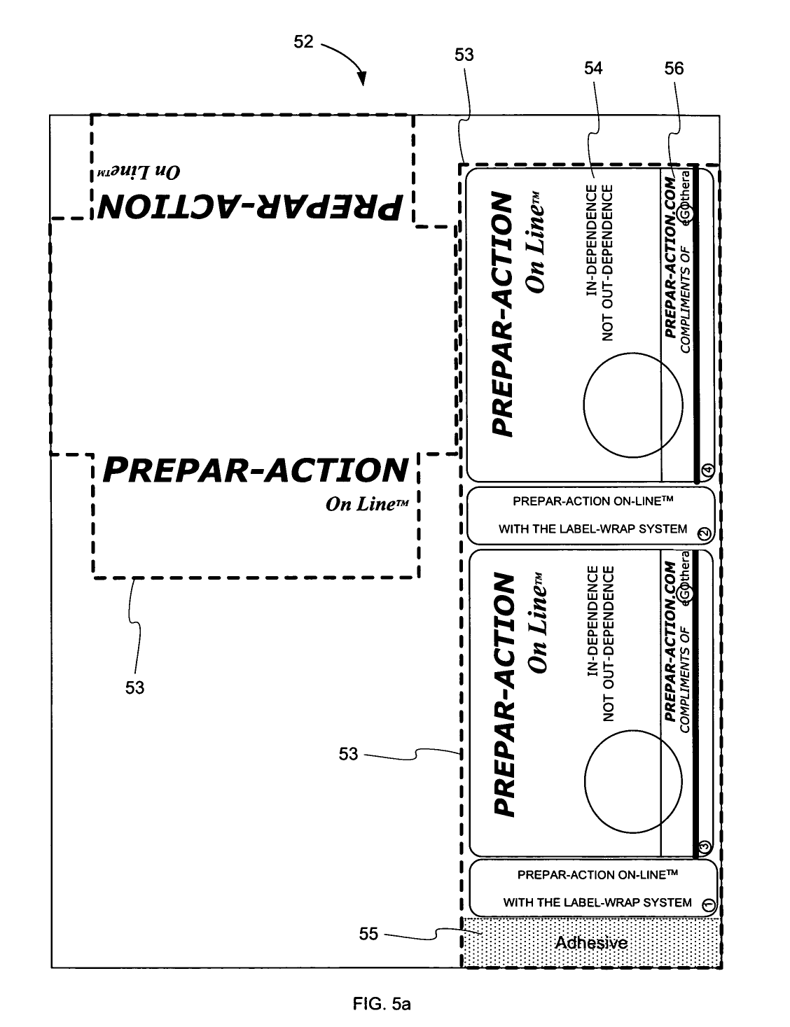

Referring to FIG. 5a, there is shown the front view of a set of cutouts (52) in accordance with still a further embodiment of the present invention, in which a replacement pack for a Canadian cigarette pack is created by cutting the cutouts (52) along the cutout lines (53) and folding the individual cutouts and using the adhesive strip (55) to form the outside covers of the cigarette pack. FIG. 5b shows the back view of the a set of cutouts (52) of FIG. 5a, though it should be noted that the reverse side is optional. Sets of cutouts for the US cigarette pack may be created in a similar manner.

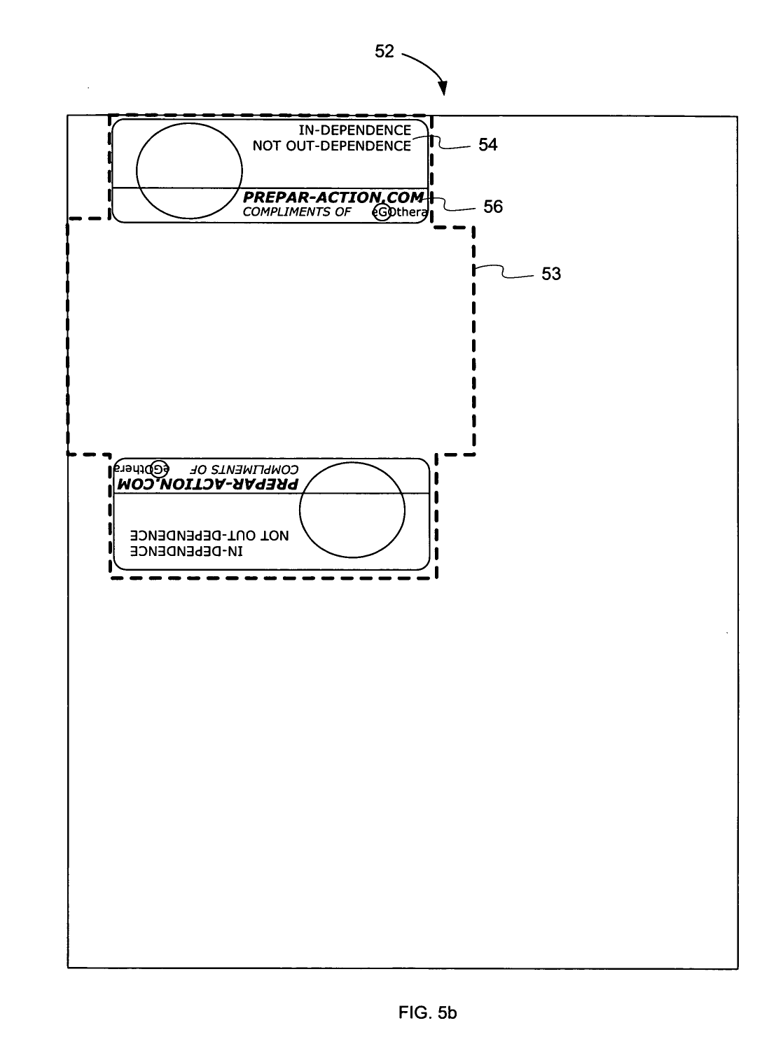

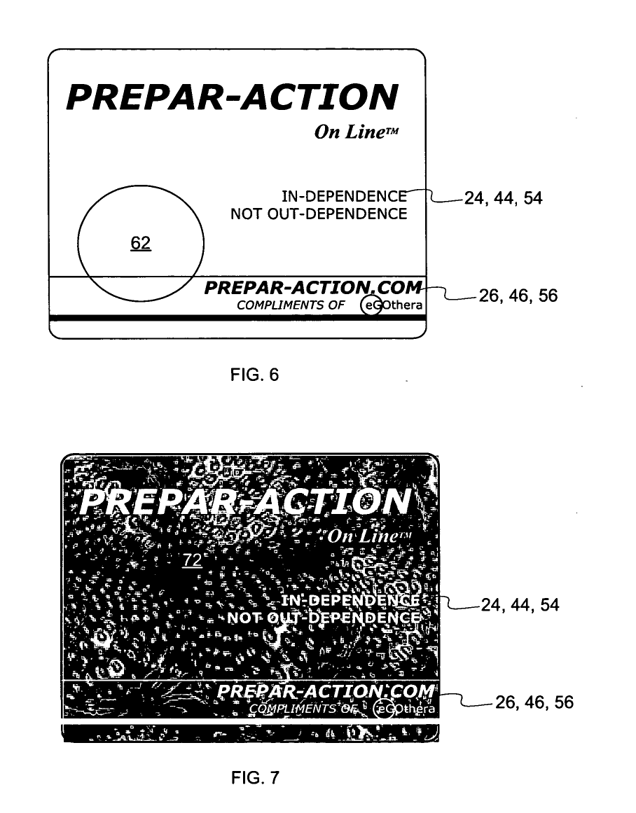

The labels (22, 42) and the replacement covers created using the set of cutouts (52) from FIG. 5a, and optionally FIG. 5b, may be adorned by one or more positive images. FIG. 6 shows an example of a simple image which may be used on the labels (22, 42) or cutouts (52), in this case a rising sun (62), in association with one or more positive messages designed to consolidate the smokers already present motivation. The messages (24, 44, 54) encourage those relationships that the smoker has chosen as important to his/her motivation for quitting smoking. FIG. 7 shows a further example of an image which may be used on the labels (22, 42) or cutouts (52), in this case strawberries (72), also in association with positive messages (24, 44, 54). The images used on the labels (22, 42) or cutouts (52) are preferably selected amongst themes or objects which are usually associated with a healthy lifestyle. To reinforce the motivational effect of the labels (22, 42) or cutouts (52), the images and the messages (24, 44, 54) may be selected so as to have a common theme.

Furthermore, other types of labels may also be used, for example business card formats with magnetic backing, or without but applicable afterwards, in order to create a series of personalized motivational refrigerator magnets in the case where substance abuse is food. The refrigerator magnets may also be die-cut in order to enhance design and visual appeal.

Although the present invention has been described by way of particular embodiments and examples thereof, it should be noted that it will be apparent to persons skilled in the art that modifications may be applied to the present particular embodiment without departing from the scope of the present invention.

1. A motivational method for helping substance abusers who have already decided to quit to overcome their addiction to an addictive substance, comprising the steps of:
a. selecting a personalized motivational message from a pre-selected group of messages posted on a web site;
b. printing said chosen motivational message; and
c. applying said printed motivational message onto an addictive substance's container;
wherein the motivational message enhances an already existing motivation for said substance abuser to quit its substance abuse.

2. A motivational method according to claim 1 wherein step (a) further comprises choosing a motivational image and step (b) further comprises printing the chosen motivational image.

3. A motivational method according to claim 2 wherein said printed motivational message includes a promotional message.

4. A motivational method according to claim 1 wherein the promotional message occupies less space than the motivational message.

5. A motivational method according to claim 1 wherein aid printed motivational message includes a sponsorship logo.

6. A motivational method according to claim 4 wherein said sponsorship logo occupies less space than said motivational message.

7. A motivational method according to claim 1 wherein the addictive substance is tobacco.

8. A motivational method according to claim 1 wherein the addictive substance is food.

9. A motivational method according to claim 1 wherein the addictive substance is alcohol.

10. A motivational method according to claim 1 wherein the addictive substance is a drug.

11. A motivational method according to claim 1 further comprising the step of:
d. repeating steps (a) to (c) every two to thirty days.

12. A motivational method according to claim 11 wherein steps (a) to (d) are repeated over a plurality of weeks

13. A motivational method according to claim 12 wherein steps (a) to (d) are repeated every three weeks.

14. A motivational method according to claim 12 wherein steps (a) to (d) are repeated every four weeks.

15. A motivational method for helping substance abusers who have already decided to quite substance abuse to overcome their addictions, comprising the steps of:
a. choosing a label comprising a personalized motivational message;
b. applying the label onto an addictive substance's container;
wherein the motivational message enhances and consolidates an already existing motivation to quit substance abuse.

16. A motivational method for helping substance abusers who have already decided to quite substance abuse to overcome their addictions, comprising the steps of:
a. choosing cut-out container comprising a personalized motivational message;
b. replacing the addictive substance's container with the cut-out container;
wherein the motivational message enhances and consolidates an already existing motivation to quit substance abuse.

17. A motivational method for helping substance abusers who have already decided to quite substance abuse to overcome their addictions, comprising the steps of:
a. choosing a personalized motivational message;
b. printing the chosen motivational message on a cut-out container; and
c. replacing the addictive substance's container with the cut-out container;
wherein the motivational message enhances and consolidates an already existing motivation to quit substance abuse.

18. A motivational method for helping substance abusers who have already decided to quite food abuse to overcome their addictions, comprising the steps of:
a. choosing a motivational message;
b. printing the chosen motivational message on a magnet; and
c. applying the magnet to a refrigerator door;
wherein the motivational message enhances and consolidates an already existing motivation to quit food abuse.

19. A web-based method for individual tailoring aid to a person wishing to cease the use of an addictive substance packaged in a container, said method comprising the steps of:
a. accessing by said person a web based server located at a remote location, said server comprising at least one group of motivational messages pre-selected by the web server administrator;
b. selecting by said person from said group of motivational messages at least one message suited to the particular needs and goals of said person;
c. printing by said person said at least one motivational message at a location selected by said person;
d. affixing by said person said printed motivational message onto said container;
such that said printed motivational message is visible to said person and wherein said motivational message enhances an already existing motivation for said person to cease using said addictive substance.

20. A system to assist a person wishing to cease the use of an addictive substance packaged in a container, said system comprising:
a. a server-based, internet accessible data base comprising at least one motivational message formatted to be selected and printed by said person so that said motivational message can be affixed to said container,
b. a communication device which allows said person to remotely access said data base,
c. a printing device on which said motivational message can be printed.

21. The system of claim 20 wherein said computing device is selected from a group comprising a personal computer, a laptop computer, a cellular phone and a personal digital assistant.
